(19) United States
(12) Patent Application Publication
(10) Pub. No.: US 2024/0091113 A1
(43) Pub. Date: Mar. 21, 2024

(54) TOPICAL SKIN MOISTURIZER SYSTEM AND METHOD OF PREPARATION

(71) Applicant: ARTHERIA HUDSON, DETROIT, MI (US)

(72) Inventor: ARTHERIA HUDSON, DETROIT, MI (US)

(21) Appl. No.: 17/977,373

(22) Filed: Oct. 31, 2022

Related U.S. Application Data
(60) Provisional application No. 63/274,479, filed on Nov. 1, 2021.

(51) Int. Cl. A61K 8/29; A61K 8/92; A61Q 19/00

(52) U.S. Cl. CPC A61K 8/29; A61K 8/922; A61Q 19/007; A61K 2800/74

(57) ABSTRACT

Disclose is a system and method of making and using a topical skin moisturizer that includes a mixture by weight of 20% to 30% shea butter, 20% to 30% mango butter, 14% to 22% marula oil, 12% to 18% kokum butter, 12% to 18% avocado oil, and 1% to 3% essential oils. The preferred embodiment within this range is about 25% shea butter, 25% mango butter, 18% marula oil, 15% kokum butter, 15% avocado oil, and 2% essential oils. The mixture is designed to be substantially solid when at room temperature and substantially liquid when warmed to about normal human body temperature. The inventive concept may include earth clays and vitamin E additions. The inventive concept may include at least one or more of titanium dioxide and zinc oxide.

### CLAIM OF PRIORITY

This application claims the benefit of and priority to U.S. Provisional Application with Ser. No. 63/274,479, filed on Nov. 1, 2021, which is incorporated herein by reference in its entirety.

### FIELD OF THE INVENTION

The inventive concept relates generally to a topical skin moisturizer system and method of preparation.

### BACKGROUND

People commonly experience discomfort from skin conditions. This discomfort can degrade their confidence and their quality of life. Many of these skin conditions are preventable with a diligent skin care regimen that includes skin care products. Many people diligent at caring for their skin, however, experience drawbacks from that care such as dryness, breakouts, flakiness, and cracking. Exacerbating the drawbacks is the prominence of water as an ingredient which displaces oil. Oil is important for maintaining moisture in the skin, and the skin is best maintained when oils removed by cleansing are replaced by other oils.

Present topical solutions may be less effective than otherwise at conditioning the skin because the ingredients are diluted by water. To achieve the same results may require using comparably more of the product by weight than required by the inventive concept. Solutions use water as a carrying agent instead of using oils as the main ingredient. Making oils and water mix creates a need for unnatural additives to achieve emulsification. Traditional lotions, therefore, include undesirable ingredients such as surfactants and emulsifiers to accommodate water as the main ingredient. Oil is the important element for moisturization. Therefore, there currently exists a need in the market for an improved topical solution for skin treatment.

### SUMMARY OF THE INVENTION

Disclose is a topical skin moisturizer that in one embodiment comprises a mixture by weight of 20% to 30% shea butter, 20% to 30% mango butter, 14% to 22% marula oil, 12% to 18% kokum butter, 12% to 18% avocado oil, and 1% to 3% essential oils. The preferred embodiment within this range is about 25% shea butter, 25% mango butter, 18% marula oil, 15% kokum butter, 15% avocado oil, and 2% essential oils. The mixture, hereafter referred to as the final mixture, is designed to be substantially solid when at room temperature and substantially liquid when warmed to about normal human body temperature.

In one embodiment of the topical skin moisturizer, the topical skin moisturizer may include at least one or more clay from a group of: fullers earth clay, Morrocan clay, bentonite clay, Rhassoul clay, and French green clay wherein up to 5% of the final mixture may be at least one or more of the clays.

In one embodiment, the topical skin moisturizer may include as a part of the final mixture 0.3% to 0.6% vitamin E, the vitamin E including a group of eight fat soluble compounds that include four tocopherols and four tocotrienols. The vitamin E may be derived from Sunflower oil in some embodiments.

In one embodiment of the topical skin moisturizer, the topical skin moisturizer may include adding titanium dioxide to the final mixture wherein a supplemented mixture includes 11.5% to 20% titanium dioxide and 80% to 88.5% the final mixture, accommodating such added ingredients to the final mixture as clays and vitamin E. Another embodiment of the topical skin moisturizer may include adding zinc oxide to the final mixture wherein the supplemented mixture includes 18.5% to 32% zinc oxide and 68% to 81.5% the final mixture, accommodating such added ingredients to the final mixture as clays and vitamin E.

In another embodiment of the topical skin moisturizer, the topical skin moisturizer comprises a mixture wherein the final mixture includes by weight 16% to 24% shea butter; 16% to 24% mango butter; 12% to 18% marula oil; 12% to 18% kokum butter; 9% to 15% avocado oil; a combined 9% to 15% at least one or more of grapeseed oil and Jojoba; 4% to 6% neem oil; and 1% to 3% essential oils.

The inventive concept now will be described more fully hereinafter with reference to the accompanying drawings, which are intended to be read in conjunction with both this summary, the detailed description, and any preferred and/or particular embodiments specifically discussed or otherwise disclosed. This inventive concept may, however, be embodied in many different forms and should not be construed as limited to the embodiments set forth herein; rather, these embodiments are provided by way of illustration only and so that this disclosure will be thorough, complete, and will fully convey the full scope of the inventive concept to those skilled in the art.

### BRIEF DESCRIPTION OF THE DRAWINGS

FIG. 1 illustrates a representative shea butter compound Vitellaria paradoxa.

FIG. 2 illustrates a representative mango butter compound Mangifera indica.

FIG. 3 illustrates a representative avocado oil compound Persea americana.

FIG. 4 illustrates a representative olive oil compound Olea europaea.

FIG. 5 illustrates a representative kokum compound Garcinia indica.

FIG. 6 illustrates a representative sunflower oil compound Helianthus.

FIG. 7 illustrates a representative jojoba compound Simmondsia chinensis.

FIG. 8 illustrates a representative xanthan powder compound.

FIG. 9 illustrates a representative marula compound Sclerocarya birrea.

FIGS. 10A-10H illustrate representative essential oil compounds.

FIG. 11 illustrates representative clays.

FIG. 12 illustrates a representative grape oil compound Vitis.

FIG. 13 illustrates a representative neem compound Azadirachta indica.

FIG. 14 illustrates a representative vitamin E compound.

FIG. 15 illustrates Titanium dioxide.

FIG. 16 illustrates Zinc oxide.

FIGS. 17A to 17C illustrate a flowchart of a representative method of making the disclosed topical skin moisturizer.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

Ranges of ingredients and temperature include the value presented as high and low endpoints of the ranges.

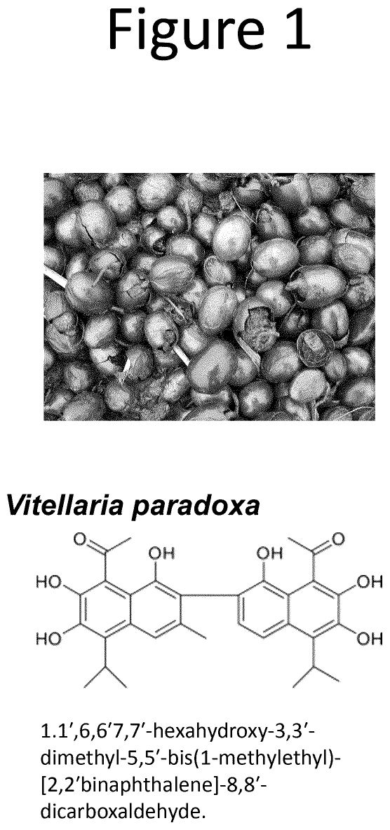

As illustrated in FIG. 1, Shea butter is a fat extracted from the nut of the African shea tree (Vitellaria paradoxa). Shea butter may be used in cosmetics as a moisturizer, salve, or lotion. The anti-inflammatory properties of shea butter may help soothe skin and relieve itching. Shea butter may be used for inflammatory skin conditions such as eczema and psoriasis.

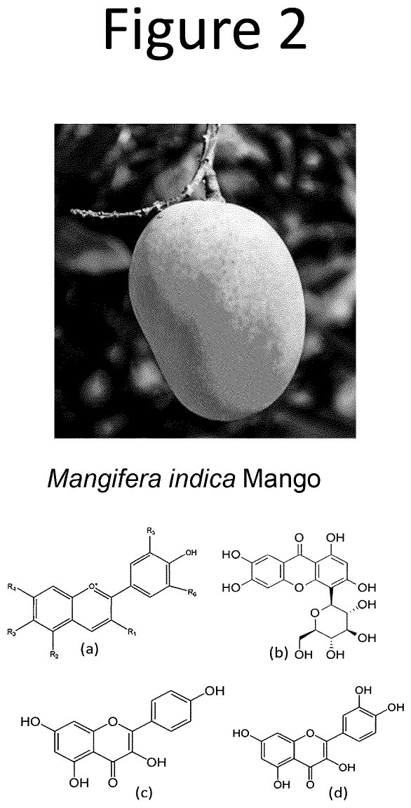

As illustrated in FIG. 2, Mango oil, mango kernel fat, or mango butter is an oil fraction obtained during the processing of mango butter. Mango oil is a seed oil extracted from the stone of the mango, the fruit of the Mangifera indica tree. The oil is semi-solid at room temperatures, but melts on contact with warm skin, with a melting point of 90-108° F. It may be used to prevent inflammation and itching caused by dry skin.

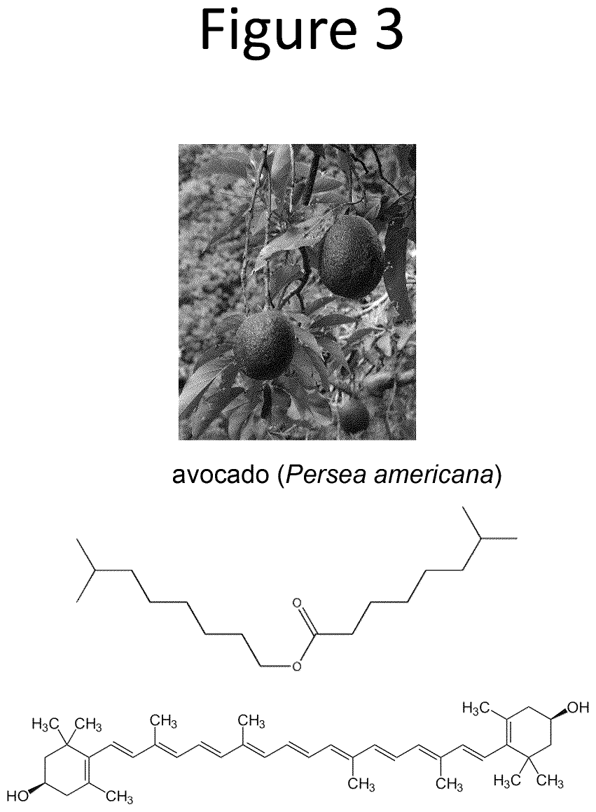

As illustrated in FIG. 3, Avocado oil is an oil extracted from the pulp of avocados, the fruit of Persea americana. It may be used for lubrication and in cosmetics. Avocado oil functions well as a carrier oil. It is high in monounsaturated fats and vitamin E and enhances the absorption of carotenoids and other nutrients. Avocado oil may be used to aid dry skin and help with skin care overall. It may be a source of vitamin E in some embodiments.

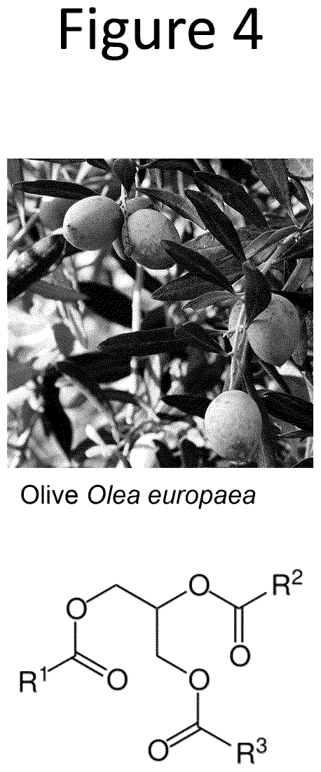

As illustrated in FIG. 4, Olive oil is a liquid fat obtained from olives (the fruit of Olea europaea; family Oleaceae). Olive oil may be used in cosmetics, pharmaceuticals, and soaps and may be used for soothing dry skin.

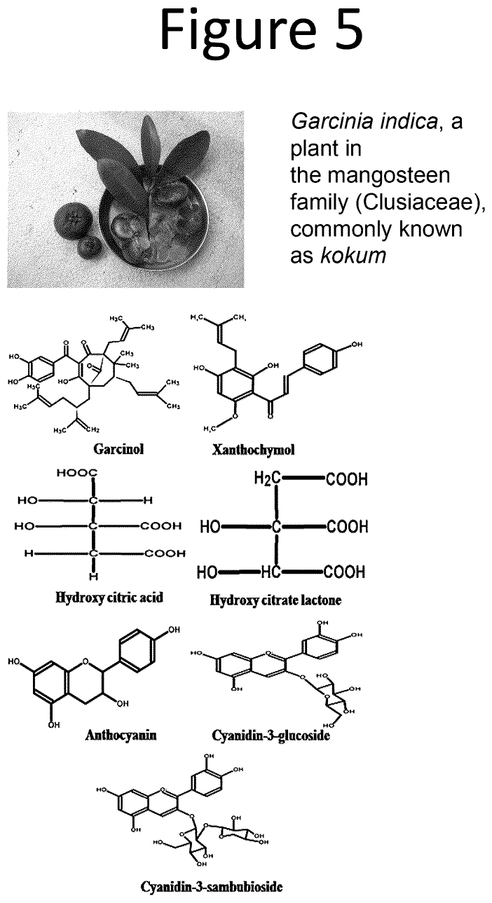

As illustrated in FIG. 5, Kokum oil is a seed oil derived from the seeds of the kokum tree (Garcinia indica; also known as wild mangosteen or red mangosteen). Kokum oil may be used in cosmetics, pharmaceuticals, and soaps. Kokum butter has emollient properties and good oxidative stability, which can assist emulsion integrity. Kokum oil melts slightly at normal human skin temperatures, making it ideal for lipsticks and balms.

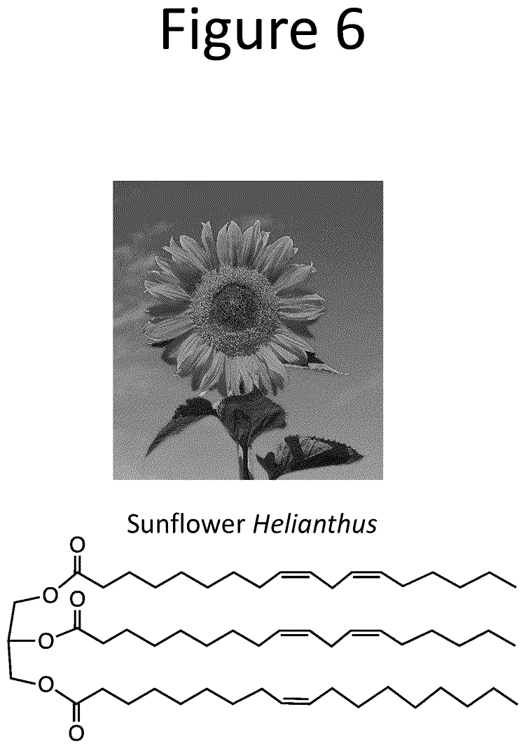

As illustrated in FIG. 6, Sunflower oil is the non-volatile oil pressed from the seeds of sunflower (Helianthus annuus). Sunflower oil may be used in cosmetic formulations. Sunflower oil is composed of linoleic acid, a polyunsaturated fat, and oleic acid, a monounsaturated fat. Sunflower oil may contain vitamin E. Sunflower oil may be applied directly to the skin for poorly healing wounds, skin injuries, psoriasis, and arthritis.

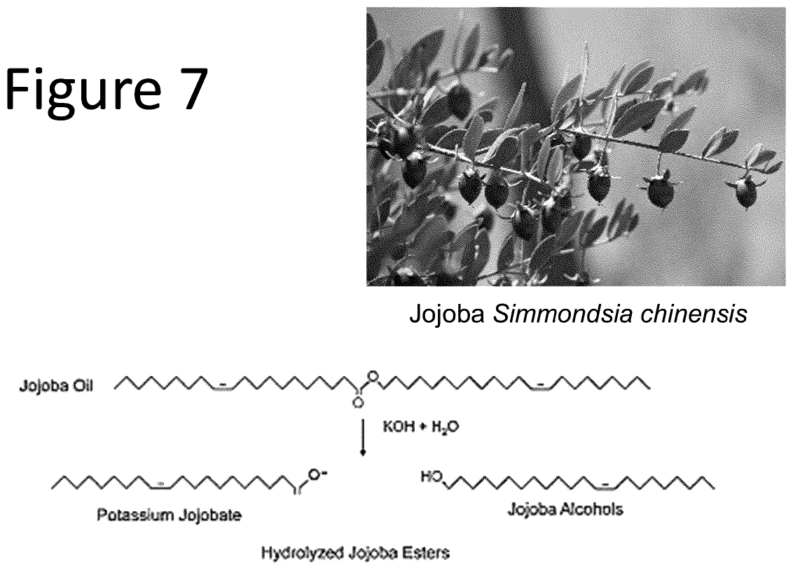

As illustrated in FIG. 7, Jojoba oil is the liquid produced in the seed of the Simmondsia chinensis jojoba plant. Jojoba oil is composed of monoesters of long-chain fatty acids and alcohols (war ester), accompanied by triglyceride esters. Triglyceride esters accounts for Jojoba oil's extreme shelf-life stability and extraordinary resistance to high temperatures as compared with most vegetable oils. Jojoba oil may be used to treat skin conditions such as acne, eczema, and psoriasis.

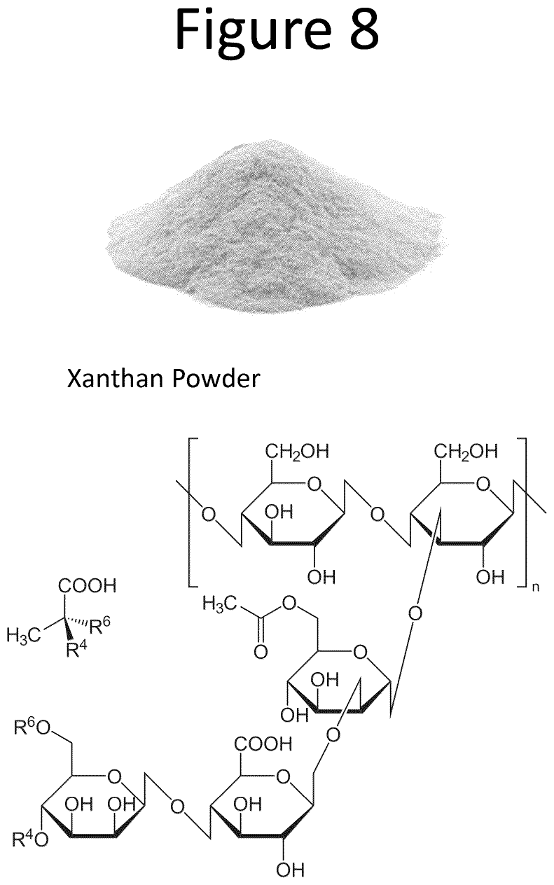

As illustrated in FIG. 8, xanthan gum is a polysaccharide with many industrial uses, including as a common food additive. It is an effective thickening agent and stabilizer to prevent ingredients from separating. Xanthan gum can be produced from simple sugars using a fermentation process and derives its name from the species of bacteria used. Xanthomonas campestris. As a thickening agent, it may be added to topical products to keep them uniformly thick.

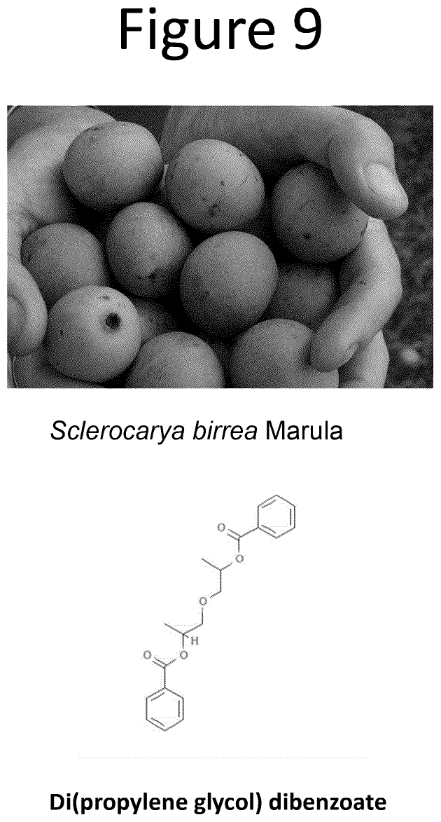

As illustrated in FIG. 9, Sclerocarya birrea “nut”, in reference to the stone inside the fleshy fruit, commonly known as the marula, is from a medium-sized deciduous tree, indigenous to the miombo woodlands of Southern Africa, the Sudano-Sahelian range of West Africa, the savanna woodlands of East Africa and Madagascar. Marula may be used for oily, acne-prone, dry, and aging skin.

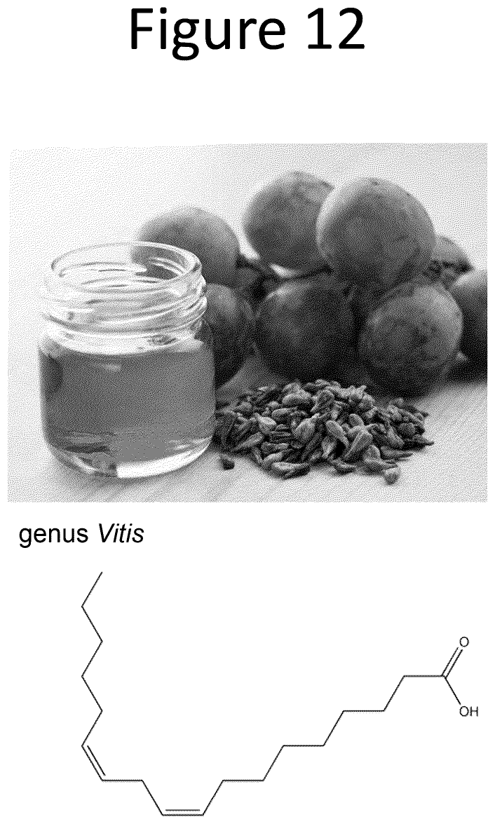

As illustrated in FIG. 12, grape seed oil (also called grapeseed oil or grape oil) is a vegetable oil derived from the seeds of grapes. Grape seed oil may be used to improve skin's elasticity and softness.

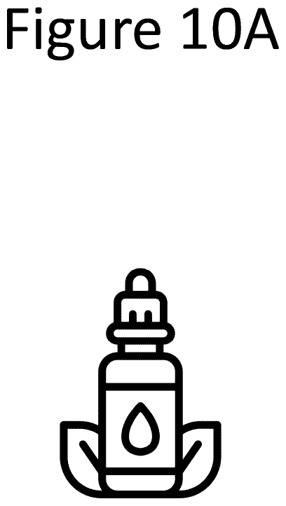

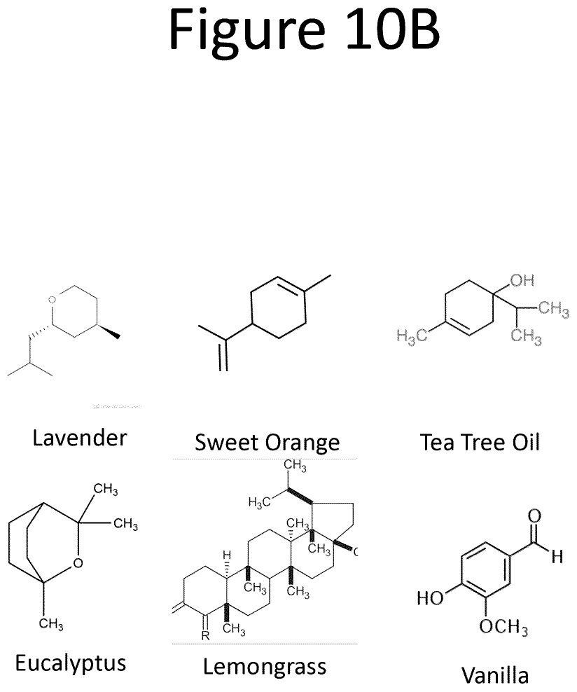

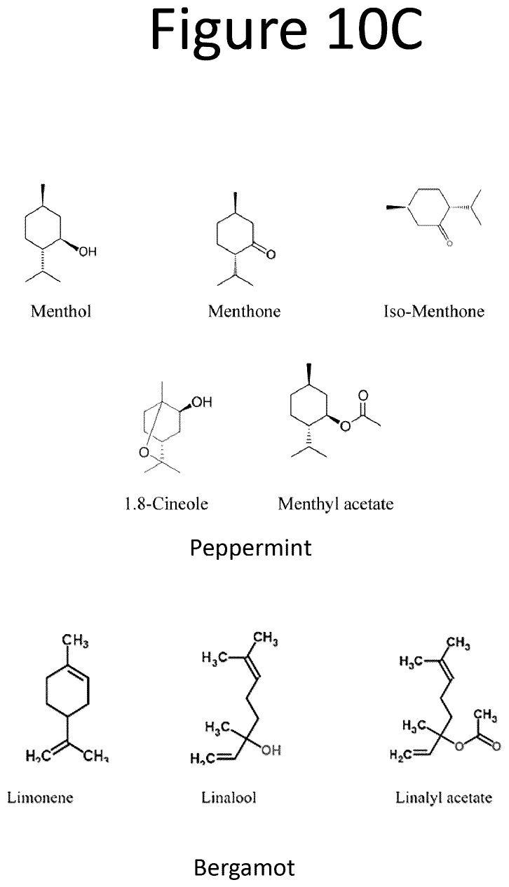

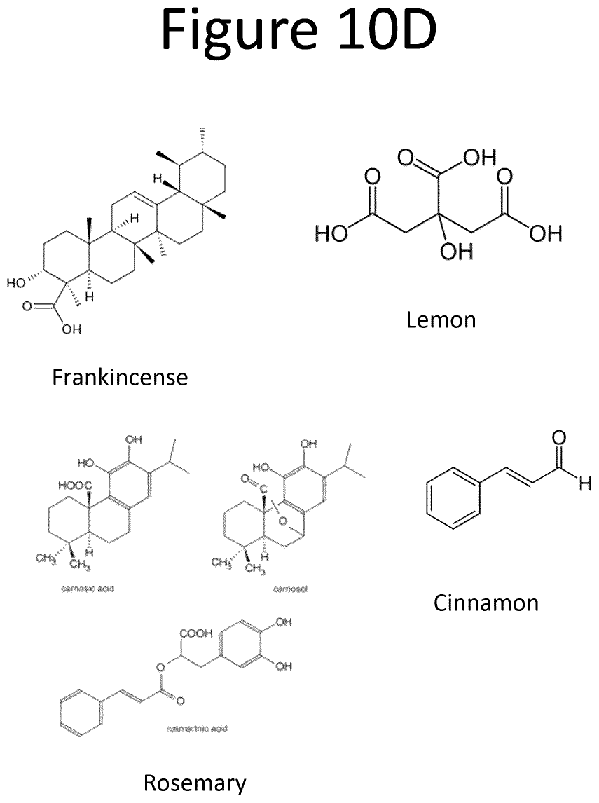

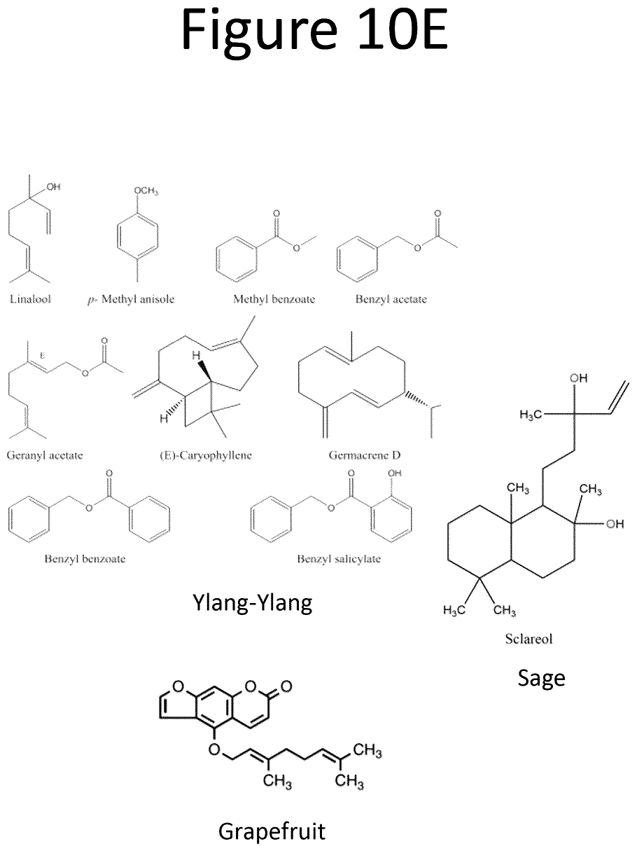

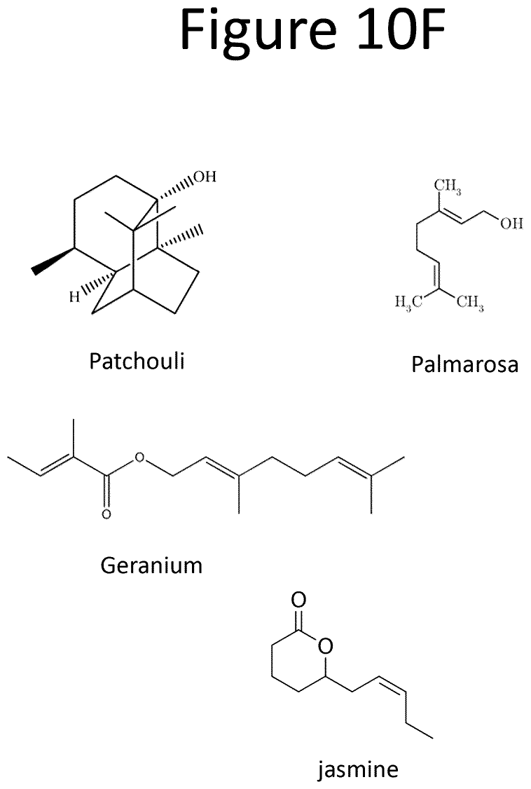

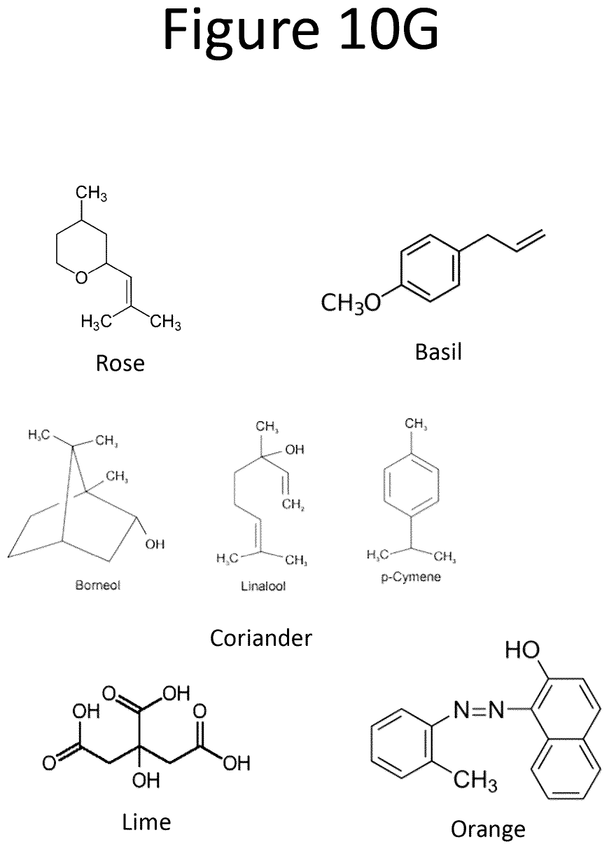

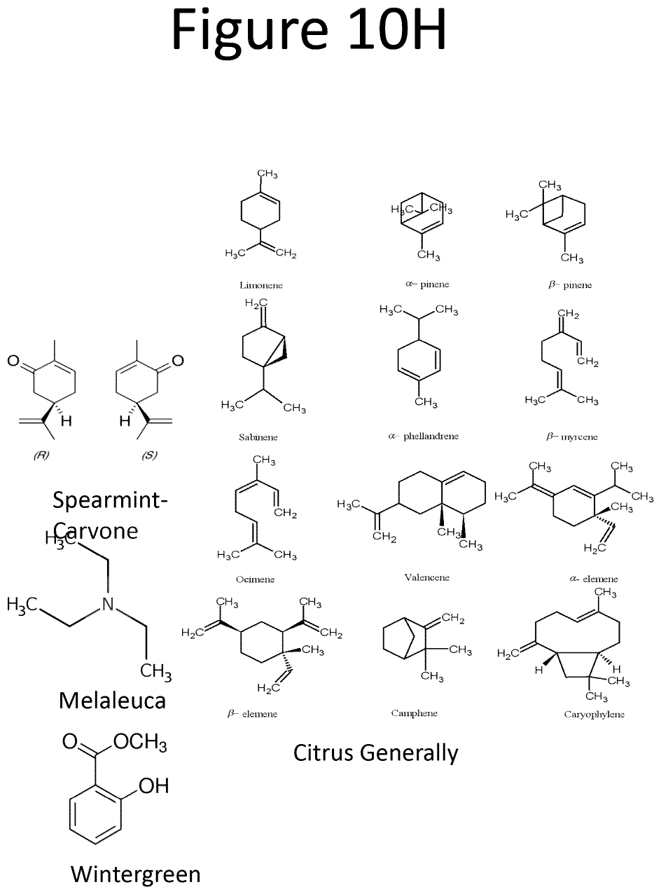

As illustrated in FIGS. 10A-10H, essential oils include at least one or more from a group of Lavender, Sweet Orange, Tea Tree, Eucalyptus, Lemongrass, Peppermint, Bergamot, Frankincense, Lemon, Rosemary, Cinnamon, Ylang-Ylang, Sage, Grapefruit, Patchouli, Palmarosa, Geranium and Cypress, jasmine, rose, vanilla, jojoba, basil, coriander, lime, orange, spearmint, tangerine, melaleuca, wintergreen, and sunflower. With their anti-inflammatory, antibacterial, and soothing properties, essential oils are used for a variety of skin care concerns. Other oils may be included as essential oils.

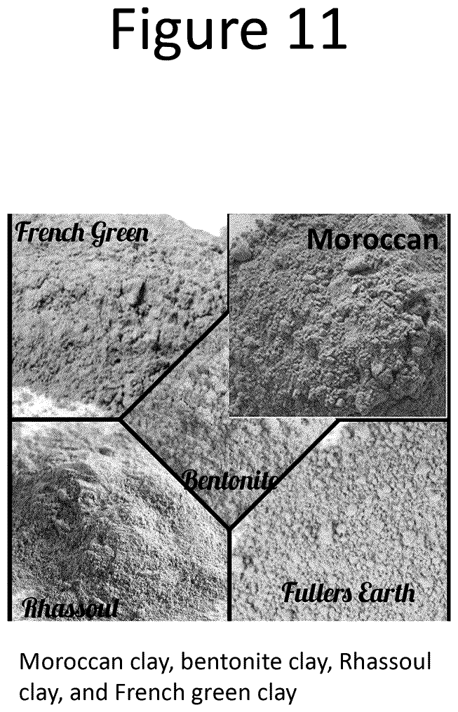

Illustrated in FIG. 11 are a wide variety of clays are used for medicinal purposes—primarily for external applications, such as the clay baths in health spas (mud therapy). Mud commonly contains nutrients, trace elements and various minerals that the body can absorb quickly. Some other benefits of mud include reducing signs of aging, improving circulation, shrinking large pores, rinsing away blackheads and helping to clear acne. Beneficial properties of clays may be had when included with other elements.

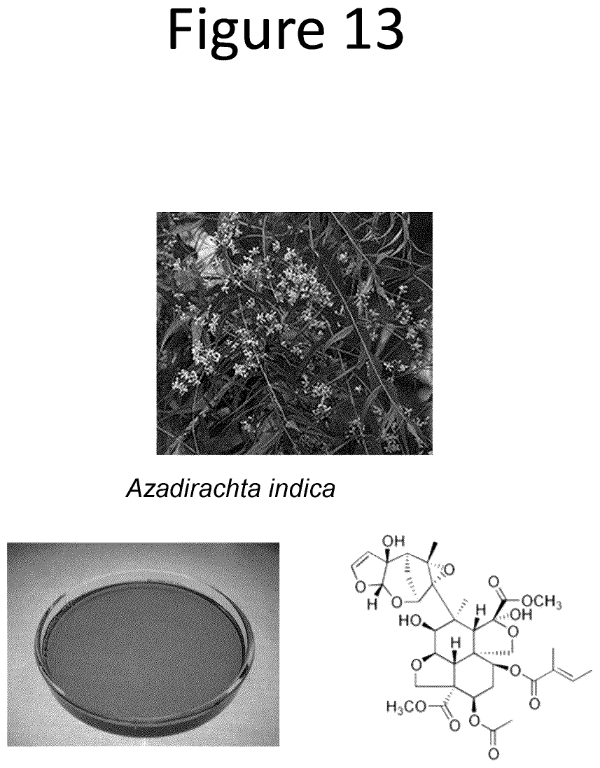

As illustrated in FIG. 13, Azadirachta indica, commonly known as neem, nimtree, or Indian lilac, and in Nigeria called dogoyaro or dogonyaro, is a tree in the mahogany family Meliaceae. It is one of two species in the genus Azadirachta and is native to the Indian subcontinent and most of countries in Africa. It is typically grown in tropical and semi-tropical regions. Its fruits and seeds are the source of neem oil. Neem oil, also known as margosa oil, is a vegetable oil pressed from the fruits and seeds of the neem. Neem oil contains fatty acids, antioxidants, and antimicrobial compounds, and these can benefit the skin in a range of ways. These compounds may help fight skin infections, promote wound healing, and combat signs of skin aging.

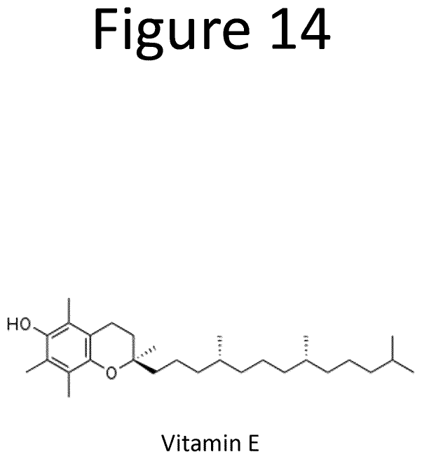

Illustrated in FIG. 14, Vitamin E is a plant-derived, lipid-soluble substance whose molecular structure is comprised of a chromanol ring with a side chain located at the C2 position. Vitamin E refers to a group of eight different compounds: α-, β-, γ-, and δ-tocopherols and the corresponding four tocotrienols. Vitamin E may help protect skin from damage caused by free radicals.

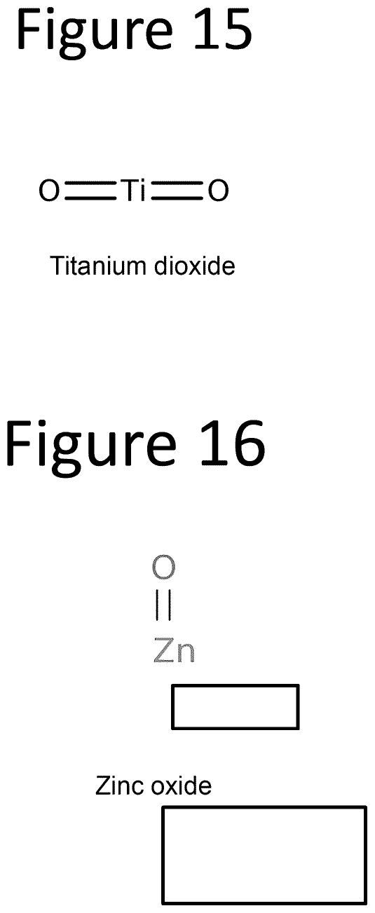

As illustrated in FIG. 15, Titanium dioxide, also known as titanium (IV) oxide or titania is the inorganic compound with the chemical formula TiO. Titanium dioxide works as a UV filtering ingredient in sunscreen—it helps protect a person's skin by blocking absorption of the sun's ultraviolet light.

As illustrated in FIG. 16, Zinc oxide is an inorganic compound with the formula ZnO. ZnO is a white powder that is insoluble in water. zinc oxide can protect skin from harmful UV rays. ZnO helps protect cells from being damaged, halts the ageing process, and prevents skin dryness by filtering out UV rays.

One representative embodiment of the topical skin moisturizer that in one embodiment comprises a mixture by weight of 16% to 24% shea butter; 16% to 24% mango butter; 12% to 18% marula oil; 12% to 18% kokum butter; 9% to 15% avocado oil; a combined 9% to 15% of at least one or more of grapeseed oil and Jojoba; 4% to 6% neem oil; and 1% to 3% essential oils. Other mixtures may be made for other embodiments. The mixture, hereafter referred to as the final mixture, is designed to be substantially solid when at room temperature and substantially liquid when warmed to about normal human body temperature.

In one representative embodiment, the topical skin moisturizer may include at least one or more clay from a group of fullers earth clay, Morrocan clay, bentonite clay, Rhassoul clay, and French green clay wherein up to 5% of the final mixture may be at least one or more of the clays.

In one representative embodiment, the topical skin moisturizer may include as a part of the final mixture 0.3% to 0.6% vitamin E, the vitamin E including a group of eight fat soluble compounds that include four tocopherols and four tocotrienols.

In one representative embodiment, the topical skin moisturizer may include adding titanium dioxide to the final mixture wherein a supplemented mixture includes 11.5% to 20% titanium dioxide and 80% to 88.5% the final mixture, accommodating such added ingredients to the final mixture as clays and vitamin E. Another embodiment of the topical skin moisturizer may include adding zinc oxide to the final mixture wherein the supplemented mixture includes 18.5% to 32% zinc oxide and 68% to 81.5% the final mixture, accommodating such added ingredients to the final mixture as clays and vitamin E.

Another embodiment of the topical skin moisturizer includes a mixture by weight of about 24% to 26% shea butter; 24% to 26% mango butter; 17% to 19% marula oil; 14% to 16% kokum butter; 14% to 16% avocado oil; 1% to 3% essential oils; and 0.3% to 0.6% vitamin E.

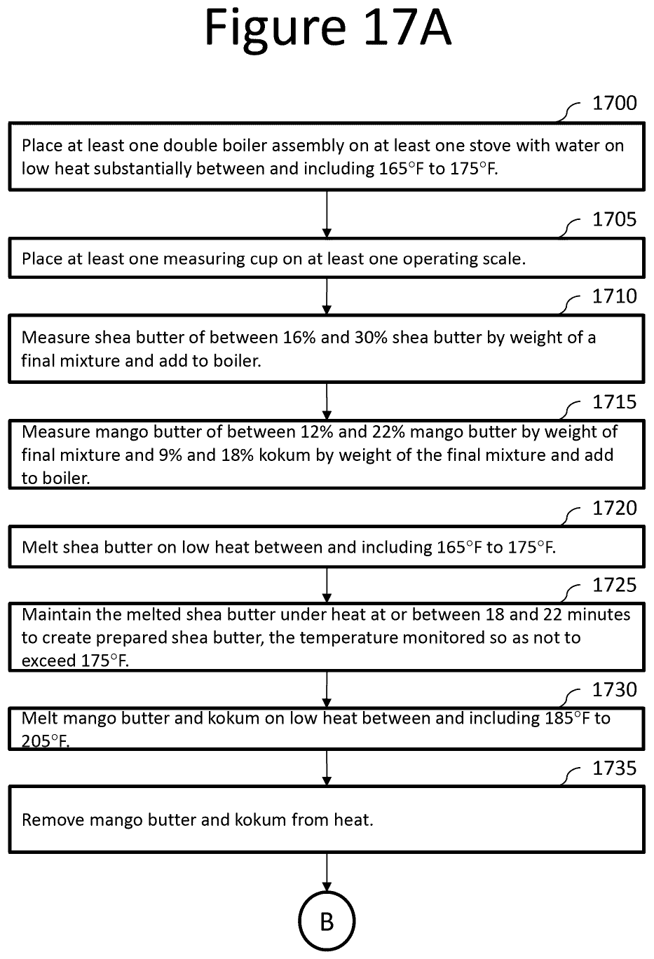

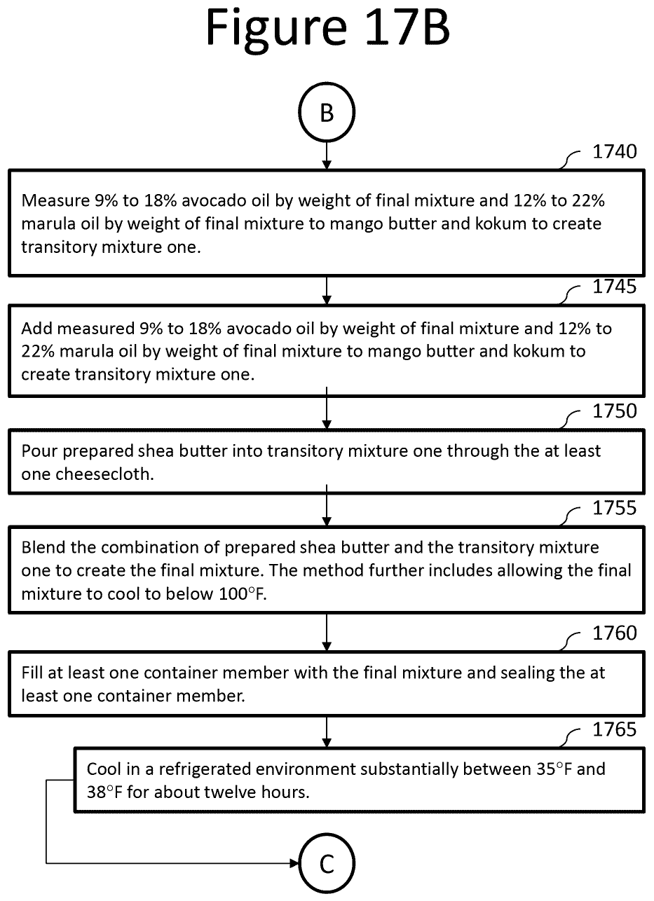

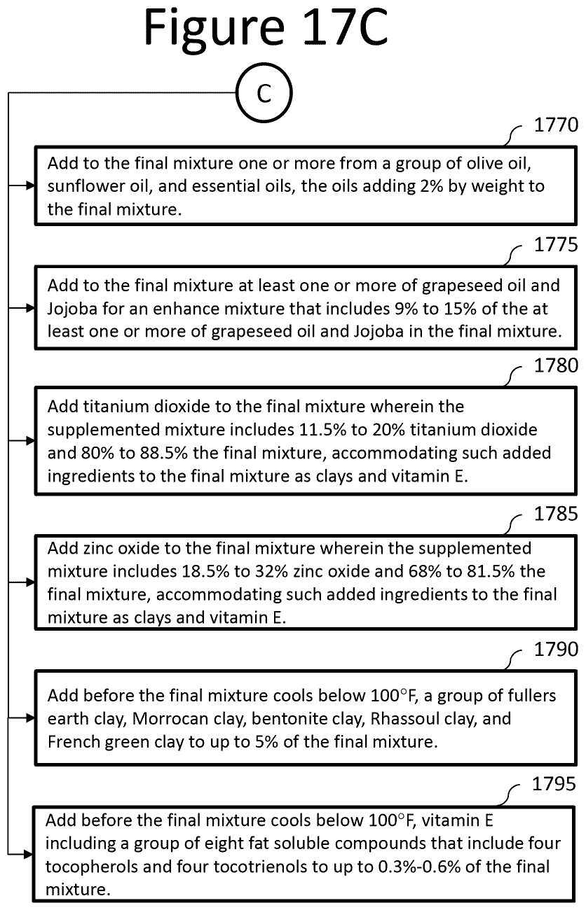

FIGS. 17A-17C illustrate a flowchart of a representative method of making a topical skin moisturizer includes the step of 1700 placing at least one double boiler assembly on at least one stove with water on low heat substantially between and including 165° F.-175° F. The method further includes the step of 1705 placing at least one measuring cup on at least one operating scale. The method further includes the step of 1710, measuring shea butter of between 16% and 30% shea butter by weight of a final mixture and adding to boiler. The method further includes the step of 1715, measuring mango butter of between 12% and 22% mango butter by weight of final mixture and 9% and 18% kokum by weight of the final mixture and add to boiler. The method further includes the step of 1720, melting shea butter on low heat between and including 165° F.-175° F. The method further includes the step of 1725, maintaining the melted shea butter under heat for between 18 and 22 minutes to create prepared shea butter, the temperature monitored so as not to exceed 175° F. The method further includes the step of 1730, melting mango butter and kokum on low heat between and including 185° F. to 205° F. The method further includes the step of 1735 removing mango butter and kokum from heat. The method further includes the step of 1740 adding measured 9% to 18% avocado oil by weight of final mixture and 12% to 22% marula oil by weight of final mixture to mango butter and kokum to create transitory mixture one. The method further includes the step of 1745 placing at least one strainer with cheesecloth over transitory mixture one and allowing transitory mixture one to cool substantially between 100° F. and 130° F. The method further includes the step of 1750 pouring prepared shea butter into transitory mixture one through the at least one cheesecloth. The method further includes the step of 1755 blending the combination of prepared shea butter and the transitory mixture one to create the final mixture. The method further includes allowing the final mixture to cool to below 100° F. The method further includes the step of 1760 filling at least one container member with the final mixture and sealing the at least one container member. The method further includes the step of 1765 cooling in a refrigerated environment substantially between 35° F. and 38° F. for about twelve hours in the preferred embodiment or as needed.

The method may further include the step of 1770, adding to the final mixture one or more from a group of olive oil, sunflower oil, and essential oils, the oils adding 2% by weight to the final mixture.

The method may further include the step of 1775, adding to the final mixture at least one or more of grapeseed oil and Jojoba for an enhance mixture that includes 9% to 15% of the at least one or more of grapeseed oil and Jojoba in the final mixture.

The method may further include the step of 1780, adding titanium dioxide to the final mixture wherein the supplemented mixture includes 11.5% to 20% titanium dioxide and 80% to 88.5% the final mixture, accommodating such added ingredients to the final mixture as clays and vitamin E.

The method may further include the step of 1785, adding zinc oxide to the final mixture wherein the supplemented mixture includes 18.5% to 32% zinc oxide and 68% to 81.5% the final mixture, accommodating such added ingredients to the final mixture as clays and vitamin E.

The method may further include the step of 1790, adding before the final mixture cools below 100° F., a group of fullers earth clay, Morrocan clay, bentonite clay, Rhassoul clay, and French green clay to up to 5% of the final mixture.

The method may further include the step of 1795, adding before the final mixture cools below 100° F., vitamin E including a group of eight fat soluble compounds that include four tocopherols and four tocotrienols to up to 0.3% to 0.6% of the final mixture.

The following patents are incorporated by reference in their entireties: U.S. Pat. Nos. 7,060,306, 8,071,138, US2016331675, US2007014881, US2021052543, US2011135746, US2017042801, U.S. Pat. No. 9,005,674, US2016045412, US2018071205, US2019209442, WO20139870, US2006172022, WO20107000, U.S. Pat. Nos. 10,137,074, 7,569,210, CN101884610b, WO20226347, U.S. Pat. No. 10,124,030, IN201611003234, US2018280274, CN109316363a, WO06010214, U.S. Pat. No. 10,045,925, US2018153793, U.S. Pat. Nos. 8,663,710, 9,561,173, EP3646848. The following papers are incorporated by reference in their entireties: Neo C MOKGOLODII, DING You-fang1, Moffat P SETSHOGO2, MA Chao1*, LIU Yu-jun1; Vegetable Oils: Composition and Analysis A. J. Dijkstra, in Encyclopedia of Food and Health, 2016; Fruit Oils: Chemistry and Functionality pp 561-5751 Cite as; The chemistry and medicinal uses of the underutilized Indian fruit tree Garcinia indica Choisy (kokum): A review, Dedication: The authors dedicate this paper to the Scientist of Central Food Technological Research Institute, Mysore, India for their initiative and pioneering studies on kokum. Author links open overlay panel Manjeshwar Shrinath Baliga Harshith P. Bhat Ramakrishna J. Pai Rekha Boloor Princy Louis Palatty.

While the inventive concept has been described above in terms of specific embodiments, it is to be understood that the inventive concept is not limited to these disclosed embodiments. Upon reading the teachings of this disclosure, many modifications and other embodiments of the inventive concept will come to mind of those skilled in the art to which this inventive concept pertains, and which are intended to be and are covered by both this disclosure and the appended claims. It is indeed intended that the scope of the inventive concept should be determined by proper interpretation and construction of the appended claims and their legal equivalents, as understood by those of skill in the art relying upon the disclosure in this specification and the attached drawings.

1. A topical skin moisturizer comprising: a mixture by weight of 16% to 24% shea butter; 16% to 24% mango butter; 12% to 18% marula oil; 12% to 18% kokum butter; 9% to 15% avocado oil; a combined 9% to 15% at least one or more of grapeseed oil and Jojoba; 4% to 6% neem oil; and 1% to 3% essential oils; and
the mixture adapted to be substantially solid when at room temperature and substantially liquid when warmed to about normal human body temperature.

2. The topical skin moisturizer of claim 1 wherein up to 5% of the mixture includes at least one or more clay from a group of: fullers earth clay, Moroccan clay, bentonite clay, Rhassoul clay, and French green clay.

3. The topical skin moisturizer of claim 1 including 0.3% to 0.6% vitamin E.

4. The topical skin moisturizer of claim 1 including adding titanium dioxide to the final mixture wherein the supplemented mixture includes 11.5% to 20% titanium dioxide and 80% to 88.5% the final mixture.

5. The topical skin moisturizer of claim 1 including adding zinc oxide to the final mixture wherein the supplemented mixture includes 18.5% to 32% zinc oxide and 68% to 81.5% the final mixture.

6. A method of making a topical skin moisturizer comprising:
placing at least one double boiler assembly on at least one stove with water on low heat substantially between and including 165° F.-175° F.;
placing at least one measuring cup on at least one operating scale;
measuring shea butter of between 16% and 30% shea butter by weight of a final mixture and add to boiler;
measuring mango butter of between 12% and 22% mango butter by weight of final mixture and 9% and 18% kokum by weight of the final mixture and add to boiler;
melting shea butter on low heat between and including 165° F.-175° F.;
maintaining the melted shea butter under heat at or between 18 and 22 minutes to create prepared shea butter, the temperature monitored so as not to exceed 175° F.;
melting mango butter and kokum on low heat between and including 185° F. to 205° F.;
removing mango butter and kokum from heat;
adding measured 9% to 18% avocado oil by weight of final mixture and 12% to 22% marula oil by weight of final mixture to mango butter and kokum to create transitory mixture one;
placing at least one strainer with cheesecloth over transitory mixture one and allowing transitory mixture one to cool substantially between 100° F. and 130° F.;
pouring prepared shea butter into transitory mixture one through the at least one cheesecloth;
blending the combination of prepared shea butter and the transitory mixture one to create the final mixture;
allowing the final mixture to cool to below 100° F.;
filling at least one container member with the final mixture and sealing the at least one container member; and
cooling in a refrigerated environment substantially between 35° F. and 38° F.

7. The method of making a topical skin moisturizer of claim 6, the method further comprising adding to the final mixture one or more from a group of olive oil, sunflower oil, and essential oils, the oils adding 2% by weight to the final mixture.

8. The method of making a topical skin moisturizer of claim 6, the method further comprising adding to the final mixture at least one or more of grapeseed oil and Jojoba, including 9% to 15% of the at least one or more of grapeseed oil and Jojoba in the final mixture.

9. The method of making a topical skin moisturizer of claim 6, the method further comprising adding titanium dioxide to the final mixture wherein the supplemented mixture includes 11.5% to 20% titanium dioxide and the final mixture includes 80% to 88.5% titanium dioxide.

10. The method of making a topical skin moisturizer of claim 6, the method further comprising adding zinc oxide to the final mixture wherein the supplemented mixture includes 18.5% to 32% zinc oxide and 68% to 81.5% the final mixture.

11. The method of making a topical skin moisturizer of claim 6, the method further comprising adding before the final mixture cools below 100° F., a group of fullers earth clay, Morrocan clay, bentonite clay, Rhassoul clay, and French green clay to up to 5% of the final mixture.

12. The method of making a topical skin moisturizer of claim 12, the method further comprising adding 0.3% to 0.6% vitamin E.

13. The method of making a topical skin moisturizer of claim 9, the method further comprising adding before the final mixture cools below 100° F., a group of fullers earth clay, Morrocan clay, bentonite clay, Rhassoul clay, and French green clay to up to 5% of the final mixture.

14. The method of making a topical skin moisturizer of claim 13, the method further comprising adding 0.3% to 0.6% vitamin E.

15. The method of making a topical skin moisturizer of claim 10, the method further comprising adding before the final mixture cools below 100° F., a group of fullers earth clay, Morrocan clay, bentonite clay, Rhassoul clay, and French green clay to up to 5% of the final mixture.

16. The method of making a topical skin moisturizer of claim 15, the method further comprising adding 0.3% to 0.6% vitamin E.

17. The method of making a topical skin moisturizer of claim 6, the method further comprising adding, before the final mixture cools below 100° F., vitamin E including a group of eight fat soluble compounds that include four tocopherols, four tocotrienols to up to 0.3% to 0.6% of the final mixture.

18. A topical skin moisturizer comprising: a mixture by weight of about 24% to 26% shea butter; 24% to 26% mango butter; 17% to 19% marula oil; 14% to 16% kokum butter; 14% to 16% avocado oil; 1% to 3% essential oils; 0.3% to 0.6% vitamin E; and
the mixture adapted to be substantially solid when at room temperature and substantially liquid when warmed to about normal human body temperature.

19. The topical skin moisturizer of claim 17 wherein up to 5% of the mixture includes at least one or more clay from a group of: fullers earth clay, Moroccan clay, bentonite clay, Rhassoul clay, and French green clay.

20. The topical skin moisturizer of claim 17 including adding at least one or more of zinc oxide and titanium dioxide to the final mixture wherein the supplemented mixture includes 11.5% to 20% of at least one or more of titanium dioxide and zinc oxide and 80% to 88.5% the final mixture.
